(19) United States
(12) Patent Application Publication
(10) Pub. No.: US 2012/0165354 A1
(43) Pub. Date: Jun. 28, 2012

(54) TREATMENT METHOD

(75) Inventors: Megan M. McLaughlin, Collegeville, PA (US); John Irving Wurzelmann, Durham, NC (US); Chun-Fang Xu, Brentford (GB)

(73) Assignee: GLAXO WELLCOME MANUFACTURING PTE LTD., Jurong (SG)

(21) Appl. No.: 13/383,935

(22) PCT Filed: Jul. 16, 2010
(86) PCT No.: PCT/US10/42211

§ 371 Date: Mar. 13, 2012

Related U.S. Application Data
(60) Provisional application No. 61/226,113, filed on Jul. 16, 2009.

(51) Int. Cl. A61K 31/506; A61P 27/02

(52) U.S. Cl. 514/275

(57) ABSTRACT

The present invention relates to methods of treating age-related macular degeneration in a patient by administration of pazopanib or pharmaceutically acceptable salts or solvates thereof.

### FIELD OF THE INVENTION

The present invention relates to methods of treating ocular neovascular disorders in a mammal. The methods comprise administering pyrimidine derivatives, benzodiazepinyl derivatives, and pharmaceutical compositions containing the same.

### BACKGROUND OF THE INVENTION

Neovascularization, also called angiogenesis, is the process of forming new blood vessels. Neovascularization occurs during normal development, and also plays an important role in wound healing following injury to a tissue. However, neovascularization has also been implicated as an important cause of a number of pathological states including, for example, cancer, rheumatoid arthritis, atherosclerosis, psoriasis, and diseases of the eye.

An eye disorder in which neovascularization plays a role is age-related macular degeneration (AMD), which is the major cause of severe visual loss in the elderly in the developed world. The vision loss in AMD results from choroidal neovascularization (CNV). The neovascularization originates from choroidal blood vessels and grows through Bruch's membrane, usually at multiple sites, into the sub-retinal pigmented epithelial space and/or the retina (see, for example, Campochiaro et al. (1999) Mol. Vis. 5:34). Leakage and bleeding from these new blood vessels with subsequent scarring and atrophy, all of which can result in vision loss.

### SUMMARY OF THE INVENTION

In one aspect of the present invention, a method of treating age-related macular degeneration in a patient suffering from such condition and having at least one Complement Factor H (CFM) Y402H polymorphism T allele includes testing the patient to determine that the patient has at least one Complement Factor H Y402H polymorphism T allele; and

administering to the patient a compound of formula (I):

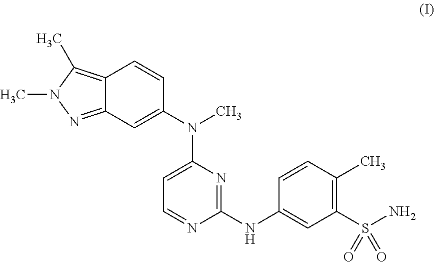

or a pharmaceutically acceptable salt or solvate thereof.

In another aspect of the present invention, a method of treating age-related macular degeneration in a patient suffering from such condition includes selecting a patient having at least one Complement Factor H (CFH) Y402H polymorphism T allele who suffers from age-related macular degeneration; and prescribing for administration to the patient a compound of formula (I):

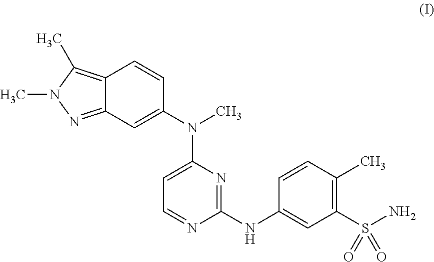

or a pharmaceutically acceptable salt or solvate thereof.

In still another aspect of the present invention, a method of treating age-related macular degeneration in a patient suffering from such condition where the patient has a Complement Factor H (CFH) Y402H polymorphism TT genotype and has never been treated for such condition by intravitreal injection includes testing the patient to determine that the patient has a Complement Factor H (CFH) Y402H polymorphism TT genotype; and administering to the patient a compound of formula (I):

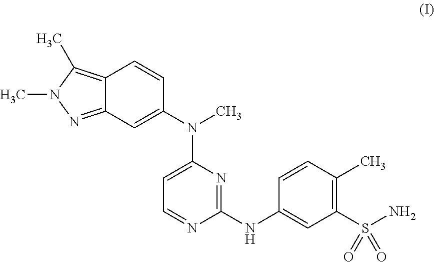

or a pharmaceutically acceptable salt or solvate thereof.

In yet a further aspect of the present invention, a method of treating age-related macular degeneration in a patient suffering from such condition where the patient has never been treated for such condition by intravireal injection includes selecting a patient having a Complement Factor H (CFH) Y402H polymorphism TT genotype who suffers from age-related macular degeneration; and prescribing for administration to the patient a compound of formula (I):

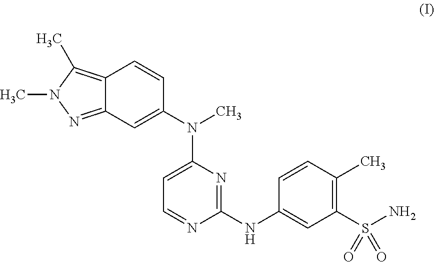

or a pharmaceutically acceptable salt or solvate thereof.

In still further aspects of the present invention, the use of a compound of formula (I):

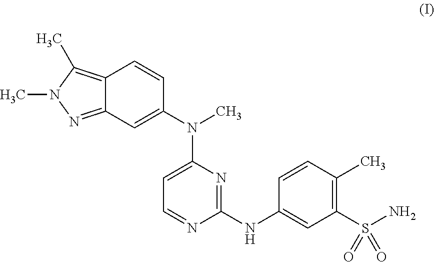

or a salt or solvate thereof for the preparation of a medicament useful in the treatment of age-related macular degeneration in a patient having at least one Complement Factor H (CFH) Y402H polymorphism T allele is described. Any of the methods claims contemplated herein encompass the corresponding use of the compound to form an appropriate medicament.

### BRIEF DESCRIPTION OF THE DRAWINGS

FIG. 1 illustrates change from baseline in central retinal thickness (CRT) at day 29 and complement factor H (CFH) Genotype following administration of pazopanib monohydrochloride at 5 and 2 mg/ml TID;

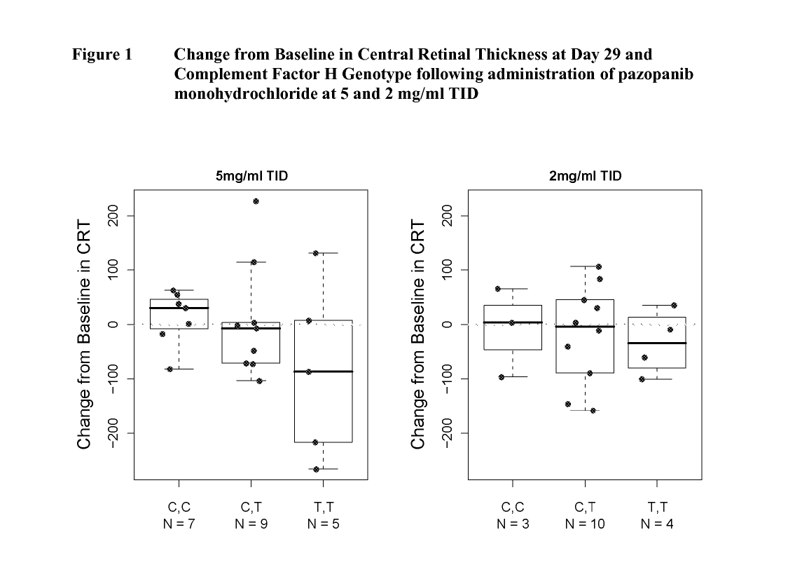

FIG. 2 illustrates change from baseline best-corrected visual acuity (BCVA) by treatment with pazopanib monohydrochloride;

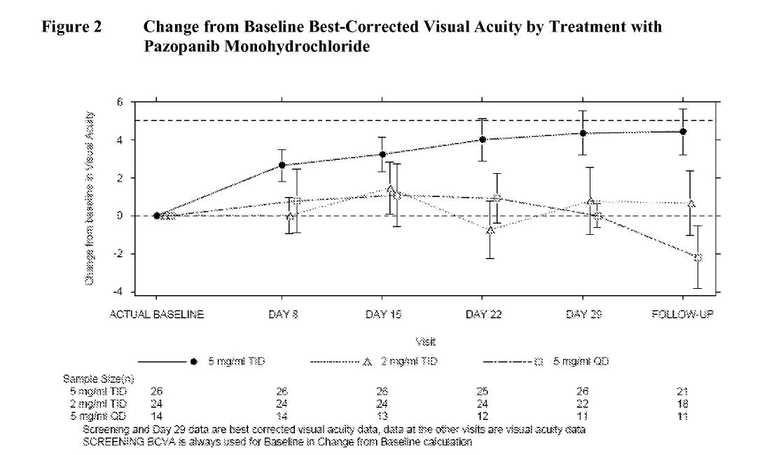

FIG. 3 illustrates change from baseline in best-corrected visual acuity (BCVA) at day 29 and complement factor H (CFH) genotype following administration of pazopanib monohydrochloride at 5 and 2 mg/ml TID;

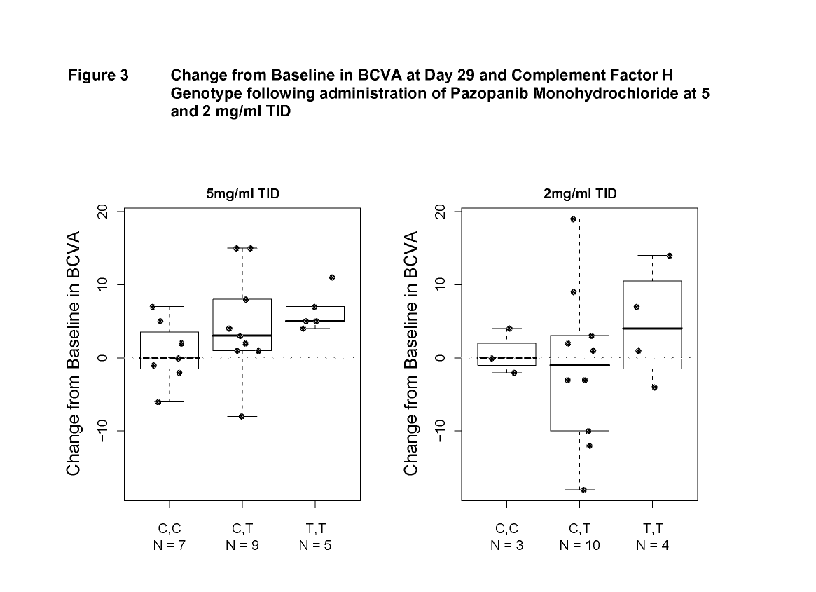

FIG. 4 illustrates distribution of change from baseline in best-corrected visual acuity (BCVA) by treatment with pazopanib monohydrochloride;

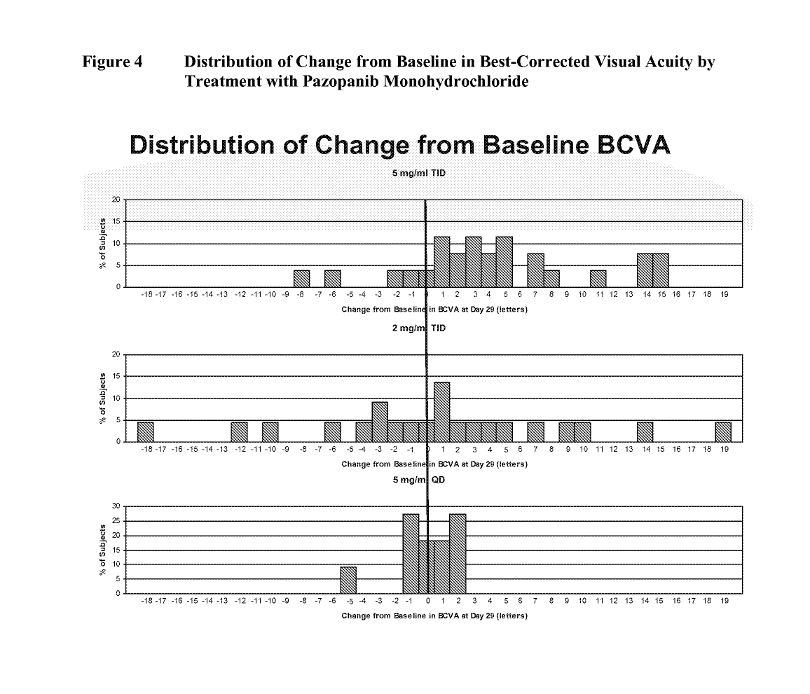

FIG. 5 illustrates change from baseline best-corrected visual acuity (BCVA) in the 5 mg/ml arm by CFH Y402H Genotype.

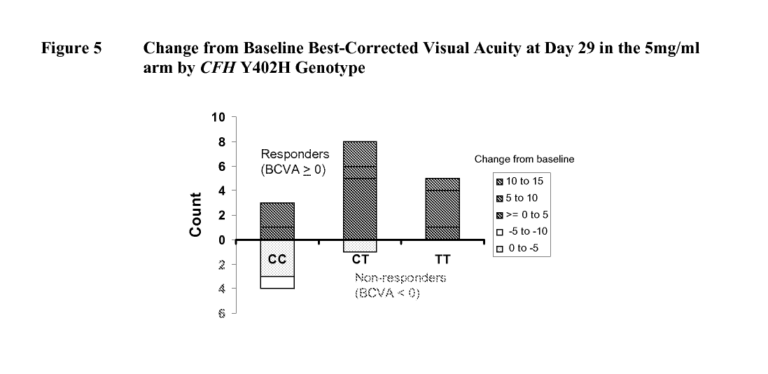

### DETAILED DESCRIPTION OF THE INVENTION

According to one aspect of the invention, a method of treating age-related macular degeneration in a patient suffering from such condition and having at least one Complement Factor H (CFH) Y402H polymorphism (rs1061170) T allele includes testing the patient to determine that the patient has at least one Complement Factor H Y402H polymorphism T allele, and administering to the patient a compound of formula (I):

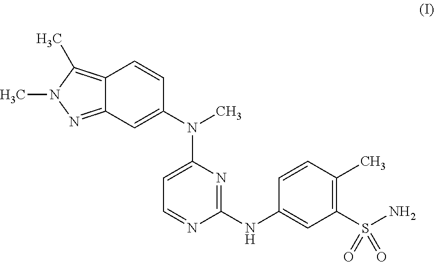

or a pharmaceutically acceptable salt or solvate thereof.

In some embodiments, the patient has the TT genotype at the CFH Y402H polymorphism. In other embodiments, the patient has the CT genotype at the CFH Y402H polymorphism.

According to a further aspect of the present invention, a method of treating age-related macular degeneration in a patient suffering from such condition where the patient has a Complement Factor H (CFH) Y402H polymorphism TT genotype and has never been treated for such condition by intravireal injection includes testing the patient to determine that the patient has a Complement Factor H (CFH) Y402H polymorphism TT genotype, and administering to the patient a compound of formula (I):

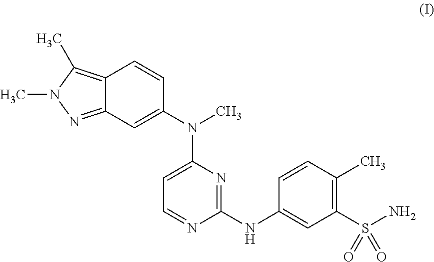

or a pharmaceutically acceptable salt or solvate thereof.

In some embodiments according to the aspects of the invention described above, the administration includes administering a pharmaceutical formulation adapted for topical administration that includes a compound of formula (I) or a pharmaceutically acceptable salt or solvate thereof. Pharmaceutical formulations for topical administration are described further herein below. In some embodiments, the pharmaceutical formulation adapted for topical administration is an eye-drop formulation. Eye-drop formulations are described further herein below. In some embodiments, the eye-drop formulation includes from a lower limit of 1, 2, 3, 4, 5, 6, 7, or 8 and an upper limit of 2, 3, 4, 5, 6, 7, 8, 9, or 10 mg of a compound of formula (I) or a pharmaceutically acceptable salt or solvate thereof per ml. In some embodiments, the eye-drop formulation includes 2 mg of a compound of formula (I) or a pharmaceutically acceptable salt or solvate thereof per ml. In other embodiments, the eye-drop formulation includes 5 mg of a compound of formula (I) or a pharmaceutically acceptable salt or solvate thereof per ml.

In some embodiments according to the aspects of the invention described above, the administration includes administering a compound of formula (I′):

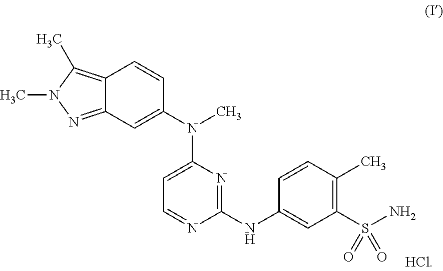

In other embodiments according to aspects of the invention described above, the administration includes administering a compound of formula (I″):

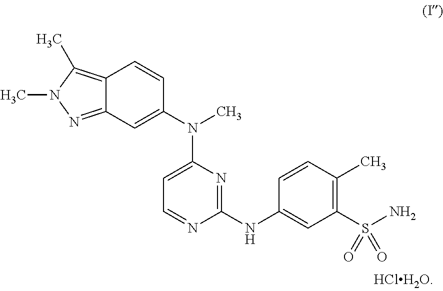

According to another aspect of the present invention, a method of treating age-related macular degeneration in a patient suffering from such condition includes selecting a patient having at least one Complement Factor H (CFH) Y402H polymorphism T allele who suffers from age-related macular degeneration, and prescribing for administration to the patient a compound of formula (I):

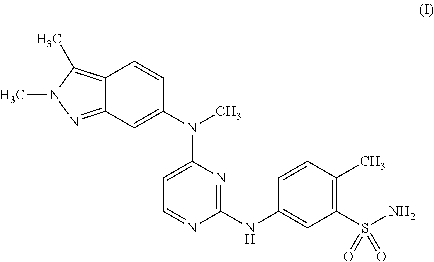

or a pharmaceutically acceptable salt or solvate thereof.

In some embodiments, the patient has the TT genotype at the CFH Y402H polymorphism. In other embodiments, the patient has the CT genotype at the CFH Y402H polymorphism.

According to still another aspect of the present invention, a method of treating age-related macular degeneration in a patient suffering from such condition where the patient has never been treated for such condition by intravireal injection includes selecting a patient having a Complement Factor H (CFH) Y402H polymorphism TT genotype who suffers from age-related macular degeneration, and prescribing for administration to the patient a compound of formula (I):

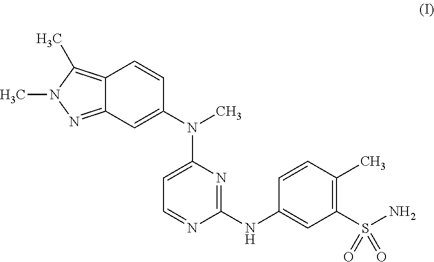

or a pharmaceutically acceptable salt or solvate thereof.

In some embodiments according to the aspects of the invention described above, the prescribing includes prescribing for administration a pharmaceutical formulation adapted for topical administration that includes a compound of formula (I) or a pharmaceutically acceptable salt or solvate thereof. Pharmaceutical formulations for topical administration are described further herein below. In some embodiments, the pharmaceutical formulation adapted for topical administration is an eye-drop formulation. Eye-drop formulations are described further herein below. In some embodiments, the eye-drop formulation includes from a lower limit of 1, 2, 3, 4, 5, 6, 7, or 8 and an upper limit of 2, 3, 4, 5, 6, 7, 8, 9, or 10 mg of a compound of formula (I) or a pharmaceutically acceptable salt or solvate thereof per ml. In some embodiments, the eye-drop formulation includes 2 mg of a compound of formula (I) or a pharmaceutically acceptable salt or solvate thereof per ml. In other embodiments, the eye-drop formulation includes 5 mg of a compound of formula (I) or a pharmaceutically acceptable salt or solvate thereof per ml.

In some embodiments according to the aspects of the invention described above, the prescribing includes prescribing for administration a compound of formula (I′):

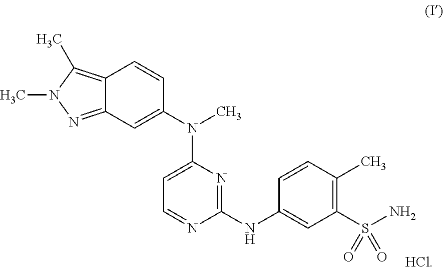

In other embodiments according to aspects of the invention described above, the prescribing includes prescribing for administration a compound of formula (I″):

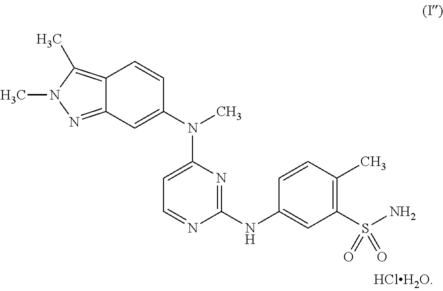

The invention provides methods for the treatment of age-related macular degeneration. As used herein, “treatment” means any manner in which one or more symptoms associated with the disorder are beneficially altered. Accordingly, the term includes healing or amelioration of a symptom or side effect of the disorder or a decrease in the rate of advancement of the disorder.

According to the methods of the invention, treatment of age-related macular degeneration (AMD) may be obtained by the administration of an effective amount of one or more therapeutic agents to the subject to be treated. As used herein, the term “effective amount” means the amount of a therapeutic agent that is sufficient to treat, prevent and/or ameliorate one or more symptoms of the disorder.

In some embodiments of methods according to the invention, the age-related macular degeneration is wet age-related macular degeneration. In other embodiments, the age-related macular degeneration is dry age-related macular degeneration. In still other embodiments, the age-related macular degeneration is late stage age-related macular degeneration.

As used herein, the term “solvate” refers to a complex of variable stoichiometry formed by a solute (in this invention, compounds of formula (I), (II), (III), or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. In particular embodiments, the solvent used is water.

The compound of formula (I):

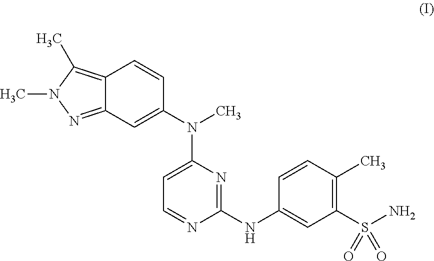

has the chemical name 5-[[4-[(2,3-dimethyl-2H-indazol-6-yl)methylamino]-2-pyrimidinyl]amino]-2-methylbenzenesulfonamide.

In certain embodiments, the salt of the compound of formula (I) is a hydrochloride salt. In a particular embodiment, the salt of the compound of formula (I) is a monohydrochloride salt as illustrated by formula (I′). The monohydrochloride salt of the compound of formula (I) has the chemical name 5-[[4-[(2,3-dimethyl-2H-indazol-6-yl)methylamino]-2-pyrimidinyl]amino]-2-methylbenzenesulfonamide monohydrochloride.

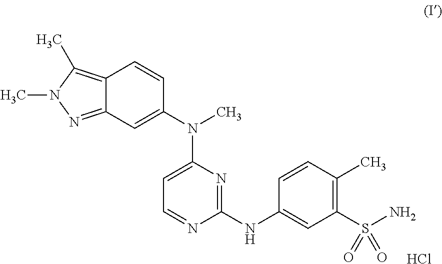

In other embodiments, the salt of the compound of formula (I) is a monohydrochloride monohydrate solvate of the compound of formula (I). The monohydrochloride monohydrate solvate of the compound of formula (I) has the chemical name 5-({4-[(2,3-dimethyl-2H-indazol-6-yl)methylamino]-2-pyrimidinyl}amino)-2-methylbenzenesulfonamide monohydrochloride monohydrate, as illustrated in formula (I″).

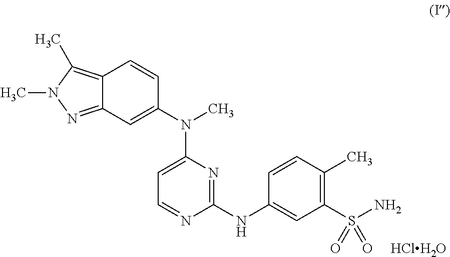

The free base, salts and solvates of the compound of formula (I) may be prepared, for example, according to the procedures of International Patent Application No. PCT/US01/49367 filed Dec. 19, 2001, and published as WO 02/059110 on Aug. 1, 2002, and International Patent Application No. PCT/US03/19211 filed Jun. 17, 2003, and published as WO 03/106416 on Dec. 24, 2003, or according the methods provided herein.

As used herein, the term “pharmaceutically acceptable salts” may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate.

The compounds used in the methods of the invention may be administered alone, or they may be administered in a pharmaceutical composition. Accordingly, the invention further provides for the use of pharmaceutical compositions in the treatment methods of the present invention. The pharmaceutical compositions include a compound of formula (I) or a pharmaceutically acceptable salt or solvate thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

Pharmaceutical formulations may be presented in unit dose forms containing a predetermined amount of active ingredient per unit dose. Such a unit may contain, for example, 1 μg to 1 g, such as 5 μg to 500 μg, 10 μg-250 μg, 0.5 mg to 700 mg, 2 mg to 350 mg, or 5 mg to 100 mg of a compound of formula (I) or salts or solvates thereof depending on the condition being treated, the route of administration and the age, weight and condition of the patient, or pharmaceutical formulations may be presented in unit dose forms containing a predetermined amount of active ingredient per unit dose. In certain embodiments, the unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The compound of formula (I) or pharmaceutically acceptable salt or solvate thereof may be administered by any appropriate route. Suitable routes include extraocular and intraocular (including, for example, intravitreal, subretinal, subscleral, intrachoroidal, and subconjuctival). It will be appreciated that the preferred route may vary with, for example, the condition of the recipient.

The methods of the present invention may also be employed in combination with other methods for the treatment of ocular neovascular disorders. In some embodiments, the methods of the invention encompass a combination therapy in which a compound of formula (I) or a pharmaceutically acceptable salt or solvate thereof is administered in conjunction with one or more additional therapeutic agents for the treatment of neovascular disorders. Non-limiting examples of additional therapeutic agents that may be used in a combination therapy include pegaptanib, ranibizumab, bevacizumab, VEGF-TRAP, PKC412, nepafenac, and integrin receptor antagonists (including vitronectin receptor agonists). See, for example, Takahashi et al. (2003) Invest. Ophthalmol. Vis. Sci. 44: 409-15, Campochiaro et al. (2004) Invest. Ophthalmol. Vis. Sci. 45:922-31, van Wijngaarden et al. (2005) JAMA 293:1509-13, U.S. Pat. No. 6,825,188 to Callahan et al., and U.S. Pat. No. 6,881,736 to Manley et al.; each of which is herein incorporated by reference for their teachings regarding these compounds.

Where a combination therapy is employed, the therapeutic agents may be administered together or separately. The same means for administration may be used for more than one therapeutic agent of the combination therapy; alternatively, different therapeutic agents of the combination therapy may be administered by different means. When the therapeutic agents are administered separately, they may be administered simultaneously or sequentially in any order, both close and remote in time. The amounts of the compound of formula (I) or a pharmaceutically acceptable salt or solvate thereof and/or the other pharmaceutically active agent or agents and the relative timings of administration will be selected in order to achieve the desired combined therapeutic effect.

Pharmaceutical formulations adapted for topical administration may be formulated as ointments, creams, suspensions, lotions, powders, solutions, pastes, gels, sprays, aerosols or oils.

For treatments of the eye, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent. Formulations to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfate anions. Embodiments of a pharmaceutical formulation of the present invention and useful in methods of the present invention are as follows:

| Component | Quantity (mg/ml) | | | |
|---|---|---|---|---|
| Product Strength | Placebo | 2 mg/ml | 5 mg/ml | Function |
| Pazopanib | NA | 2.167 | 5.417 | Active |
| Monohydrochloride | | | | |
| B-cyclodextrin | 70.00 | 70.00 | 70.00 | Solubility |
| sulfobutylether | | | | Enhancer |
| (Captisol) | | | | |
| Monobasic Sodium | 3.450 | 3.450 | 3.450 | Buffering |
| Phosphate | | | | Agent |
| Sodium Chloride | 1.685 | 1.685 | 1.461 | Tonicity |
| | | | | Modifier |
| Water for injection | q.s. | q.s. | q.s. | Solvent |
| Hydrochloric Acid | As needed | As needed | As needed | pH Adjustment |
| Sodium Hydroxide | As needed | As needed | As needed | pH Adjustment |

These formulations are presented as a preservative free, single use eye drop formulations. Current fill per container is 0.45 mL with a drop weight of ˜40 μL. Density of solution is 1.03 mg/mL. Osmolality is ˜290 mOs m/kg. The pH of the formulations is 5. These formulations were used in obtaining the results detailed in the Biological section herein. These formulations can be modified to have a pH down to a value of 4. These formulations can also be modified to have a Captisol concentration up to 10% w/v.

In some embodiments of the present invention, the pharmaceutical formulations are adapted for intraocular administration by means of intraocular injection or other device for ocular delivery. Examples of ocular devices that may be used in the methods of the invention include periocular or intravitreal devices, contact lenses and liposomes. See, for example, U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; 6,299,895; 6,331,313; 6,416,777; 6,649,184; 6,719,750; 6,660,960; and U.S. Patent Publication Nos. 2003/0064088, 2004/0247645, and, 2005/0113806; each of which is herein incorporated by reference for purposes of their teachings of optical devices.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein is includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

According to the methods of the invention, a compound of formula (I) or a pharmaceutically acceptable salt or solvate thereof is administered or prescribed to a patient. The amount of administered or prescribed compound will depend upon a number of factors including, for example, the age and weight of the patient, the precise condition requiring treatment, the severity of the condition, the nature of the formulation, and the route of administration. Ultimately, the amount will be at the discretion of the attendant physician.

In some embodiments of the methods of the invention, the total amount of the compound of formula (I) or a pharmaceutically acceptable salt or solvate thereof administered or prescribed to be administered per day can be 1 μg to 10 mg. In other embodiments, such amount can be 5 μg to 500 μg. In still other embodiments, such amount can be 10 μg-250 μg. In some embodiments, the compound of formula (I) or a pharmaceutically acceptable salt or solvate thereof is administered or prescribed to be administered one, two, three, four, or more times per day. In some embodiments, the compound of formula (I) or a pharmaceutically acceptable salt or solvate thereof is administered or prescribed to be administered by administering one, two, three, four or more drops of a suitable pharmaceutical formulation one, two, three, four, or more times per day. In some embodiments, the suitable pharmaceutical formulation comprises between a lower limit of 1, 2, 3, 4, 5, 6, 7, 8, or 9 and an upper limit of 2, 3, 4, 5, 6, 7, 8, 9, or 10 mg of a compound of formula (I) or a pharmaceutically acceptable salt or solvate thereof per ml.

The following examples are intended for illustration only and are not intended to limit the scope of the invention in any way.

### EXAMPLES

### Clinical Experience in Age-Related Macular Degeneration (AMD) Patients

### Design and Demographics

Three dosing regimens of pazopanib monohydrochloride eye drops (5 mg/mL TID, 2 mg/mL TID, 5 mg/mL QD) were administered for 28 days to 70 subjects with occult or minimally classic subtypes of choroidal neovascularization due to AMD. This study was designed to measure pharmacological activity of topically administered pazopanib in target tissues (choroid and retina) of patients with AMD by weekly evaluation of central retinal lesion thickness as measured by optical coherence tomography (OCT). In addition, the ocular and systemic safety and systemic pharmacokinetics of pazopanib treatment for 28 days were evaluated. Evaluation of efficacy was performed on an exploratory basis by weekly measurement of visual acuity, performing best-corrected visual acuity assessments at screening and day 29.

Of the total of 70 subjects that were randomized, 28 had been previously treated with anti-angiogenic agents for treatment of macular degeneration and 42 were treatment-naïve. The protocol excluded subjects who had received any treatment for AMD within 60 days of the first dose of study medication in this trial. A total of 68 subjects completed all study assessments, of which 63 completed the assessments of Day 29 visit without receiving any rescue medication. 6 subjects received anti-VEGF rescue medication prior to the Day 29 assessment (1 of 27 in the 5 mg/ml TID arm, 2 of 27 in the 2 mg/ml TID, 3 of 16 subjects in the 5 mg QD arm, which was discontinued per protocol amendment due to feasibility.) Baseline characteristics in the study were similar between treatment groups, with the exception of age and lesion size, which were increased in the 2 mg/ml TID arm.

TABLE 1
Baseline Characteristics

| | 5 mg/mL TID | | 2 mg/mL TID | | 5 mg/mL QD | |
|---|---|---|---|---|---|---|
| | N = 27 | | N = 27 | | N = 16 | |
| Number of Subjects | | | | | | |
| Age in Years, Mean (Range) | 72.6 | (57-87) | 76.5 | (64-88) | 71.5 | (60-83) |
| Sex, n (%) | | | | | | |
| Female: | 20 | (74%) | 14 | (52%) | 9 | (56%) |
| Male: | 7 | (26%) | 13 | (48%) | 7 | (44%) |
| BMI, Mean (Range) | 29.1 | (20-51) | 27.5 | (20-44) | 24.7 | (22-29) |
| Height, (Mean and Range) | 160.6 | (127-183) | 166.4 | (151-188) | 165.8 | (150-182) |
| Weight, (Mean and Range) | 73.9 | (45-104) | 76.5 | (54-118) | 67.8 | (57-82) |
| Ethnicity, n (%) | | | | | | |
| Hispanic or Latino: | 0 | | 2 | (7%) | 1 | (6%) |
| Not Hispanic or Latino: | 27 | (100%) | 25 | (93%) | 15 | (94%) |
| Race, n (%) | | | | | | |
| White—White/Caucasian/European Heritage | 27 | (100%) | 27 | (100%) | 16 | (100%) |
| Baseline Total Lesion Size, (Mean; n) | 8.3 | | 10.3 | | 8.7 | |
| | n = 26 | | n = 24 | | n = 14 | |
| Baseline BCVA, (Mean; n) | 61.3 | | 62.0 | | 61.9 | |
| | n = 26 | | n = 24 | | n = 14 | |
| Baseline CRLT, (Mean; n) | 446.2 | | 463.4 | | 433.1 | |
| | n = 26 | | n = 24 | | n = 14 | |
| CNV Type, (Mean and %) | n = 27 | | n = 26 | | n = 16 | |
| No CNV | 1 | (4%) | 1 | (4%) | 2 | (13%) |
| Classic CNV | 3 | (11%) | 1 | (4%) | 1 | (6%) |
| Classic & Occult | 3 | (11%) | 6 | (23%) | 3 | (19%) |
| Occult | 20 | (74%) | 18 | (69%) | 9 | (56%) |

| | | | | |
|---|---|---|---|---|
| Discoform Scar | 0 | 0 | 1 | (6%) |

### Optical Coherence Tomography

Sites were certified by a central reading center to collect images to assess Central Retinal Thickness (CRT, subretinal fluid) and Central Retinal Lesion Thickness (CRLT, subretinal fluid and neovascular lesion) on a Stratus OCT 3 instrument weekly. Values were then determined by manual measurement at the reading center. As used herein, “OCT CRLT” means optical coherence tomography central retinal/lesion thickness as determined by manual measurement of the distance between the inner and outer retina, inclusive of subretinal fluid and any choroidal neovascularization (CNV) as measured in the central 1 mm area of the 7 mm posterior pole scan. As used herein, “OCT CRT” means optical coherence tomography central retinal thickness as determined by manual measurement of the distance between the inner and outer retina inclusive of any subretinal fluid as measured in the central 1 mm area of the 7 mm posterior pole scan. In the overall population, no statistically significant changes from baseline were observed in these patients. However, statistically significant decreases in CRT (mean decrease of 89 micron at day 29) were observed in subjects with the CFH TT genotype who had received 5 mg/ml TID. A similar trend of was seen in subjects receiving 2 mg/ml TID (not statistical significant).

TABLE 2
Change from Baseline in Central Retinal Thickness at Day
29 and Complement Factor H Genotype following administration
of pazopanib monohydrochloride at 2 and 5 mg/ml TID

| Treatment | | | CRT LSMean | | Two-sided | One-sided |
|---|---|---|---|---|---|---|
| Regimen | Genotype | N | (SE) | | P-value | P-value |
| 2 mg/ml TID | CC | 3 | 22.15 | (48.14) | 0.6482 | 0.6759 |
| | CT | 10 | −14.6 | (25.89) | 0.5758 | 0.2879 |
| | TT | 4 | −45.5 | (41.07) | 0.2756 | 0.1378 |
| 5 mg/ml TID | CC | 7 | 9.23 | (30.95) | 0.7673 | 0.6164 |
| | CT | 9 | 21.51 | (27.73) | 0.4432 | 0.7784 |
| | TT | 5 | −88.8 | (36.61) | 0.0206 | 0.0103 |

### Visual Acuity

Sites were trained and certified for visual acuity assessment with the standard procedure developed for the Early Treatment Diabetic Retinopathy Study (ETDRS) and adapted for the Age Related Eye Disease Study (AREDS). Best-corrected visual acuity (BCVA) measurements were performed at screening and Day 29 visits and, at other visits, use of the previous refraction for the visual acuity (VA) assessment was allowed. Statistically significant increases in visual acuity were observed in the 5 mg/ml TID group only and this was enhanced in the previously treated group and those with a T allele.

TABLE 3
Statistical Analysis of Change from Baseline in BCVA Day 29

| | | | | | | |
|---|---|---|---|---|---|---|
| Treatment | N | n | Point Estimate | SE | 95% CI | P-Value |

Overall

| | | | | | | |
|---|---|---|---|---|---|---|
| 5 mg/mL TID | 26 | 26 | 4.32 | 1.290 | (1.74, 6.91) | 0.0015 |
| 2 mg/mL TID | 24 | 22 | 0.76 | 1.403 | (−2.05, 3.57) | 0.5921 |
| 5 mg/mL QD | 14 | 11 | 0.09 | 1.988 | (−3.89, 4.07) | 0.9646 |

TABLE 4
Statistical Analysis of Change from Baseline in BCVA at Day 29 by
Sub-Populations (Previously Treated, Treatment Naïve)

| | | | | Point | | | |
|---|---|---|---|---|---|---|---|
| Group | Treatment | N | n | Estimate | SE | 95% CI | P-value |
| Naïve | 5 mg/mL TID | 15 | 15 | 3.38 | 1.734 | (−0.09, 6.86) | 0.0563 |
| Subjects | 2 mg/mL TID | 14 | 13 | 1.16 | 1.859 | (−2.57, 4.89) | 0.5355 |
| | 5 mg/mL | 8 | 6 | 0.80 | 2.738 | (−4.70, 6.29) | 0.7721 |
| | once daily | | | | | | |
| Previously | 5 mg/mL TID | 11 | 11 | 5.61 | 2.033 | (1.53, 9.69) | 0.0079 |
| Treated | 2 mg/mL TID | 10 | 9 | 0.18 | 2.236 | (−4.31, 4.67) | 0.9360 |
| Subjects | 5 mg/mL | 6 | 5 | −0.80 | 3.022 | (−6.86, 5.26) | 0.7925 |
| | once daily | | | | | | |

TABLE 5
Change from Baseline BCVA at Day 29 by CFH Y402H genotypes

| Treatment | | | BCVA LSMean | Two-sided | One-sided |
|---|---|---|---|---|---|
| Regimen | Genotype | N | (SE) | P-value | P-value |
| 2 mg/ml TID | CC | 3 | 0.85 (4.07) | 0.8359 | 0.4179 |
| | CT | 10 | −1.52 (2.24) | 0.5015 | 0.7493 |
| | TT | 4 | 5.43 (3.60) | 0.1404 | 0.0702 |
| 5 mg/ml TID | CC | 7 | 0.36 (2.68) | 0.8947 | 0.4473 |
| | CT | 9 | 4.09 (2.38) | 0.0947 | 0.0473 |
| | TT | 5 | 6.96 (3.18) | 0.0355 | 0.0178 |

These results suggest that the treatment effect of pazopanib monohydrochloride eye drops on retinal thickness is limited mainly to the patient population that carries the TT genotype of CFH and the visual acuity effect is limited to those that have a CFH T allele (CT or TT) at the doses administered. As a comparison, CC genotype patients have been shown to also respond less well to bevacizumab and ranibizumab, but the OCT impact of these drugs at the general population level is easily detectable and therefore more pronounced than in this study.

The observed results initially appear atypical, with visual acuity results reaching statistical significance in the overall patient population, (an effect as pronounced as ranibizumab), when the PD read-out (retinal thickness by OCT) did not. The explanation for this VA-OCT dissociation is unclear, but could be due to a novel additional mechanism possibly linked to inhibition of RTKs beyond VEGFR, and independent of retinal thickness. An alternative explanation, however, is that the robust VA and OCT responses from subjects with the CFH TT genotype are diluted when assessed in the overall population; the expected VA-OCT concordance emerges in the pharmacogenetic analysis.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method of treating age-related macular degeneration in a patient suffering from such condition and having at least one Complement Factor H (CFH) Y402H polymorphism T allele, said method comprising:
testing said patient to determine that said patient has at least one Complement Factor H Y402H polymorphism T allele; and
administering to said patient a compound of formula (I): 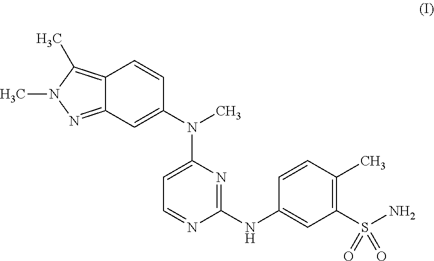
or a pharmaceutically acceptable salt or solvate thereof.

2. The method according to claim 1, wherein said patient has the TT genotype at the CFH Y402H polymorphism.

3. The method according to claim 1, wherein said patient has the CT genotype at the CFH Y402H polymorphism.

4. The method according to claim 1, wherein said administration comprises administering a pharmaceutical formulation adapted for topical administration comprising a compound of formula (I) or a pharmaceutically acceptable salt or solvate thereof.

5. The method according to claim 4, wherein the pharmaceutical formulation adapted for topical administration is an eye-drop formulation.

6. The method according to claim 5, wherein said administration comprises administering from one to three drops of the eye-drop formulation, one to four times per day.

7. The method according to claim 5, wherein the eye-drop formulation comprises from 1 to 8 mg of a compound of formula (I) or a pharmaceutically acceptable salt or solvate thereof per ml.

8. The method according to claim 7, wherein the eye-drop formulation comprises 2 mg of a compound of formula (I) or a pharmaceutically acceptable salt or solvate thereof per ml.

9. The method according to claim 7, wherein the eye-drop formulation comprises 5 mg of a compound of formula (I) or a pharmaceutically acceptable salt or solvate thereof per ml.

10. The method according to claim 1, wherein the administration comprises administering a compound of formula (I′):

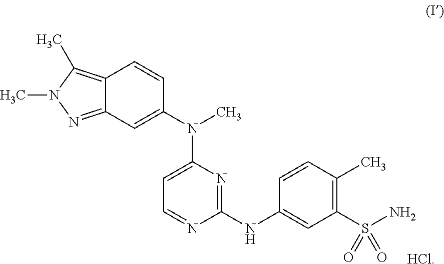

11. The method according to claim 1, wherein the administration comprises administering a compound of formula (I″):

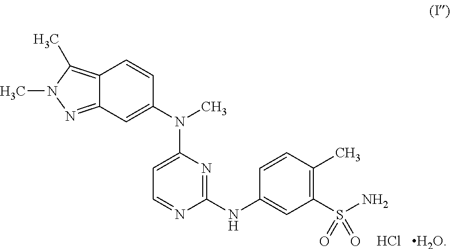

12. A method of treating age-related macular degeneration in a patient suffering from such condition, said method comprising:
selecting a patient having at least one Complement Factor H (CFH) Y402H polymorphism T allele who suffers from age-related macular degeneration; and
prescribing for administration to said patient a compound of formula (I): 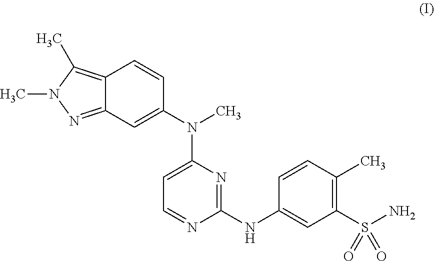
or a pharmaceutically acceptable salt or solvate thereof.

13. The method according to claim 12, wherein said patient has the TT genotype at the CFH Y402H polymorphism.

14. The method according to claim 12, wherein said patient has the CT genotype at the CFH Y402H polymorphism.

15. The method according to claim 12, wherein said prescribing comprises prescribing for administration a pharmaceutical formulation adapted for topical administration comprising a compound of formula (I) or a pharmaceutically acceptable salt or solvate thereof.

16. The method according to claim 15, wherein the pharmaceutical formulation adapted for topical administration is an eye-drop formulation.

17. The method according to claim 16, wherein said prescribing comprises prescribing for administration from one to three drops of the eye-drop formulation, one to three times per day.

18. The method according to claim 16, wherein the eye-drop formulation comprises from 1 to 10 mg of a compound of formula (I) or a pharmaceutically acceptable salt or solvate thereof per ml.

19. The method according to claim 18, wherein the eye-drop formulation comprises 2 mg of a compound of formula (I) or a pharmaceutically acceptable salt or solvate thereof per ml.

20. The method according to claim 18, wherein the eye-drop formulation comprises 5 mg of a compound of formula (I) or a pharmaceutically acceptable salt or solvate thereof per ml.

21. The method according to claim 12, wherein said prescribing comprises prescribing a compound of formula (I′):

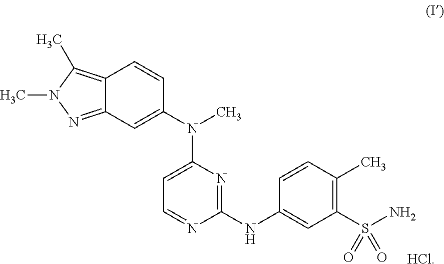

22. The method according to claim 12, wherein said prescribing comprises prescribing a compound of formula (I″):

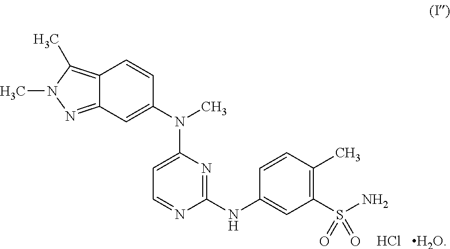

23. A method of treating age-related macular degeneration in a patient suffering from such condition, said patient having a Complement Factor H (CFH) Y402H polymorphism TT genotype and never having been treated for such condition by intravitreal injection, said method comprising:
testing said patient to determine that said patient has a Complement Factor H (CFH) Y402H polymorphism TT genotype; and
administering to said patient a compound of formula (I): 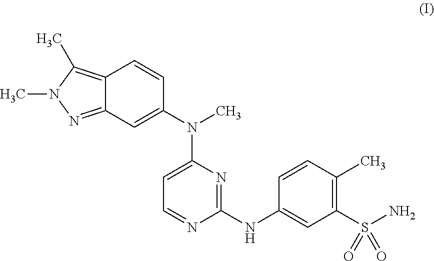
or a pharmaceutically acceptable salt or solvate thereof.

24. The method according to claim 23, wherein said administration comprises administering a pharmaceutical formulation adapted for topical administration comprising a compound of formula (I) or a pharmaceutically acceptable salt or solvate thereof.

25. The method according to claim 24, wherein the pharmaceutical formulation adapted for topical administration is an eye-drop formulation.

26. The method according to claim 25, wherein said administration comprises administering from one to three drops of the eye-drop formulation, one to three times per day.

27. The method according to claim 25, wherein the eye-drop formulation comprises from 1 to 10 mg of a compound of formula (I) or a pharmaceutically acceptable salt or solvate thereof per ml.

28. The method according to claim 27, wherein the eye-drop formulation comprises 2 mg of a compound of formula (I) or a pharmaceutically acceptable salt or solvate thereof per ml.

29. The method according to claim 27, wherein the eye-drop formulation comprises 5 mg of a compound of formula (I) or a pharmaceutically acceptable salt or solvate thereof per ml.

30. The method according to claim 23, wherein the administration comprises administering a compound of formula (I′):

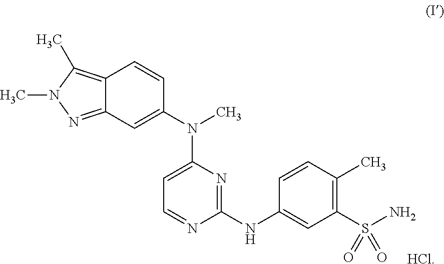

31. The method according to claim 23, wherein the administration comprises administering a compound of formula (I″):

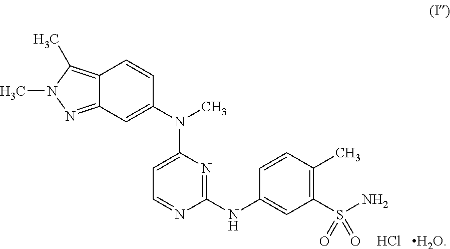

32. A method of treating age-related macular degeneration in a patient suffering from such condition, said patient never having been treated for such condition by intravireal injection, said method comprising:
selecting a patient having a Complement Factor H (CFH) Y402H polymorphism TT genotype who suffers from age-related macular degeneration; and
prescribing for administration to said patient a compound of formula (I): 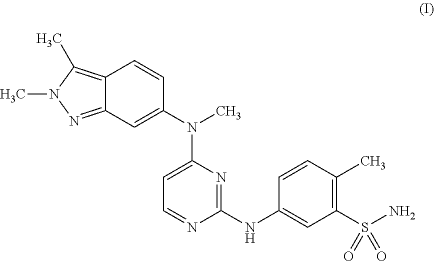
or a pharmaceutically acceptable salt or solvate thereof.

33. The method according to claim 32, wherein said prescribing comprises prescribing for administration a pharmaceutical formulation adapted for topical administration comprising a compound of formula (I) or a pharmaceutically acceptable salt or solvate thereof.

34. The method according to claim 33, wherein the pharmaceutical formulation adapted for topical administration is an eye-drop formulation.

35. The method according to claim 34, wherein said prescribing comprises prescribing for administration from one to three drops of the eye-drop formulation, one to three times per day.

36. The method according to claim 34, wherein the eye-drop formulation comprises from 1 to 10 mg of a compound of formula (I) or a pharmaceutically acceptable salt or solvate thereof per ml.

37. The method according to claim 36, wherein the eye-drop formulation comprises 2 mg of a compound of formula (I) or a pharmaceutically acceptable salt or solvate thereof per ml.

38. The method according to claim 36, wherein the eye-drop formulation comprises 5 mg or a compound of formula (I) or a pharmaceutically acceptable salt or solvate thereof per ml.

39. The method according to claim 32, wherein said prescribing comprises prescribing for administration a compound of formula (I′):

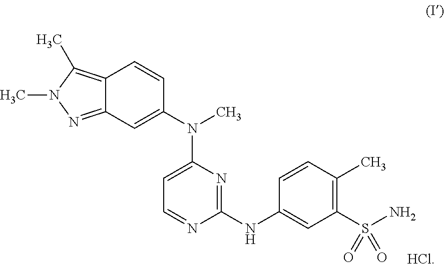

40. The method according to claim 32, wherein said prescribing comprises prescribing for administration a compound of formula (I″):

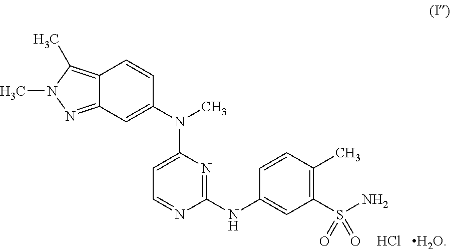

41-52. (canceled)
